(19) United States
(12) Patent Application Publication
(10) Pub. No.: US 2011/0130796 A1
(43) Pub. Date: Jun. 2, 2011

(54) Thermostimulation methods using multilayer pads with integrated temperature regulation

(76) Inventor: Mohn Louise, London (GB)

(21) Appl. No.: 12/592,470

(22) Filed: Nov. 27, 2009

(51) Int. Cl. A61F 7/08; A61N 1/00; A61B 5/053

(52) U.S. Cl. 607/3; 607/96; 600/547

(57) ABSTRACT

A therapeutic method. The inventive method includes the steps of: applying a pad to a patient having a biometric sensor adapted to feedback a signal; coupling said feedback signal to an inline control system; coupling said inline control system to a console; and regulating energy applied to said pad by the inline control system based on the output of said sensor coupled to said inline control system and an output of said console. In the illustrative embodiment, the therapeutic method is thermostimulation and includes the steps of applying heat and stimulation via the pad, sensing temperature at the pad with an embedded temperature sensor and regulating the heat current at the pad via the inline control system in response to the output of the temperature sensor. In an alternative embodiment, the embedded sensor is a galvanic skin response sensor for measuring skin conductivity and having output is used by the inline controller in the treatment of dermatological conditions, relaxation treatment, desensitization training or other purposes. In another embodiment, the embedded sensor is electromyography sensor for measuring muscular electrical activity for the treatment of patients for weakness, impaired muscle strength, or gait analysis. In yet another embodiment, the embedded sensor is a pulse sensor. In this case, the inline controller uses the data to measure the patient's heart rate. In any case, useful data with respect to the performance of and conditions at each pad is displayed either on the inline controller and/or at the console. In addition, the inline controllers may be adapted to communicate with each other and/or with other external devices.

### BACKGROUND OF THE INVENTION

1. Field of the Invention

The present invention relates to therapeutic methods. More specifically, the present invention relates to methods for providing electrical and thermal stimulation.

2. Description of the Related Art

For a variety of therapeutic applications, several treatment modalities are currently known in the art including electrical stimulation, heat therapy and thermostimulation. Electrical stimulation involves the application of an electrical current to a single muscle or a group of muscles. The resulting contraction can produce a variety of effects from strengthening injured muscles and reducing edema to relieving pain and promoting healing. Typical electrical stimulation systems are limited to two to four channels and therefore allow only two to four pads to be applied to a patient. The pads are usually quite small and typically powered with a battery. This results in the application of a small amount of power and a low treatment depth of the resulting electric field. The shallow depth of the electric field generated by conventional electrical stimulation systems limits performance and patient benefit. Some systems have attempted to address this limitation by applying more current, often from a line or main supply source. However, the small size of conventional electrical stimulation pads is such that on the application of larger amounts of power, i.e. the use of higher currents, patients often report the experience of pain or discomfort.

Heat therapy or thermal stimulation itself is very useful as it has a number of effects such as relaxation of muscle spasm and increased blood flow that promotes healing. However, combination therapy, i.e. the synergistic use of other modalities such as massage, ultrasound and/or electrical stimulation has been found to be more effective than heat therapy alone.

Thermostimulation is one such combination therapy that involves the use of heat therapy and electrical stimulation simultaneously. With thermostimulation, the healing benefits of heat are provided along with the strengthening, toning, pain relieving and healing benefits of electrical stimulation. Moreover, the application of heat has been found effective in that it allows the patient to tolerate higher currents. This yields higher electric fields strengths, greater depths of penetration and therefore, more positive results than could be achieved with electrical stimulation without heat.

Unfortunately, there are several problems associated with conventional thermostimulation systems. One problem is due to poor or inadequate pad design. That is, conventional pads are small, hard and die cut with sharp flat edges. The rectangular shape of the pads does not conform to the natural shape of muscle tissue. In addition, conventional pads tend to exhibit a current fall off over the length of the pad. This limits the performance of conventional pads. Further, the connectors are subject to detachment and therefor often fail to comply with government requirements in certain countries. (See for example EN standard 60601-2-35 for medical electrical devices.)

Further, conventional thermostimulation pads are not waterproof. As a consequence, sweat from the patient combined with the pad gel can cause the stimulation connector and press studs to short directly to the patient, which can result in the patient being shocked or burned.

Moreover, conventional thermostimulation pads are generally inflexible and yield to breakage of the heating element if bent or folded too frequently. More significantly, conventional thermostimulation pads are not designed to detect, measure and/or monitor temperature. Consequently, effective temperature regulation is not provided with conventional thermostimulation systems.

Copending U.S. patent applications entitled THERMOSTIMULATION SYSTEM INCLUDING MULTILAYER PADS WITH INTEGRATED TEMPERATURE REGULATION, filed ______ by L. Mohn (Atty. Docket No. Luzmon-1), THERMOSTIMULATION PAD WITH INTEGRATED TEMPERTURE SENSOR, filed ______ by L. Mohn (Atty. Docket No. Luzmon-6), and INLINE CONTROL SYSTEM FOR THERAPEUTIC PAD, filed ______ by L. Mohn (Atty. Docket No. Luzmon-7), the teachings of each of which are incorporated herein by reference, disclose and claim a novel system and apparatus for thermostimlation using both thermal and electrical stimulation in inline control and pads with temperature feedback.

Hence, a need remains in the art for new therapeutic methods that leverage the capabilities of the system and apparatus taught by Mohn in the above-noted applications.

### SUMMARY OF THE INVENTION

The need in the art is addressed by the therapeutic method of the present invention. In a most general embodiment, the inventive method includes the steps of: applying a pad to a patient having a biometric sensor adapted to feedback a signal; coupling said feedback signal to an inline control system; coupling said inline control system to a console; and regulating energy applied to said pad by the inline control system based on the output of said sensor coupled to said inline control system and an output of said console.

Many implementations are enabled by the present teachings. In the illustrative embodiment, the therapeutic method is thermostimulation and includes the steps of applying heat and stimulation via the pad, sensing temperature at the pad with an embedded temperature sensor and regulating the heat current at the pad via the inline control system in response to the output of the temperature sensor.

In an alternative embodiment, the embedded sensor is a galvanic skin response sensor for measuring skin conductivity and having output is used by the inline controller in the treatment of dermatological conditions, relaxation treatment, desensitization training or other purposes.

In another embodiment, the embedded sensor is electromyography sensor for measuring muscular electrical activity for the treatment of patients for weakness, impaired muscle strength, or gait analysis.

In yet another embodiment, the embedded sensor is a pulse sensor. In this case, the inline controller uses the data to measure the patient's heart rate.

In any case, useful data with respect to the performance of and conditions at each pad is displayed either on the inline controller and/or at the console. In addition, the inline controllers may be adapted to communicate with each other and/or with other external devices.

### BRIEF DESCRIPTION OF THE DRAWINGS

FIG. 1 is a perspective view of a typical thermostimulation system implemented in accordance with conventional teachings.

FIG. 2 is a simplified block diagram of a typical thermostimulation electrical system provided within the console of FIG. 1.

FIG. 3 is a simplified perspective view of a thermostimulation system implemented in accordance with an illustrative embodiment of the teachings of the above-noted copending application entitled THERMOSTIMULATION SYSTEM INCLUDING MULTILAYER PADS WITH INTEGRATED TEMPERATURE REGULATION incorporated herein by reference.

FIG. 4 shows a perspective bottom view of the pad of FIG. 3.

FIG. 5 is an exploded upside down view of a portion of the pad of FIG. 3 in disassembled relation.

FIG. 6 is a perspective side view of the inline control system of FIG. 3 fully assembled.

FIG. 7 is a perspective side view of the inline control system of FIG. 6 disassembled.

FIG. 8 is an electrical block diagram of the system described in copending U.S. patent application entitled THERMOSTIMULATION SYSTEM INCLUDING MULTILAYER PADS WITH INTEGRATED TEMPERATURE REGULATION the teachings of which have been incorporated herein by reference.

FIG. 9 is a flow diagram of the firmware executed by the main microcontroller of FIG. 8.

FIG. 10 is a flow diagram of the firmware executed by the safety microcontroller of FIG. 8.

FIG. 11 is a flow diagram of the firmware executed by the main and safety microcontrollers of FIG. 8 for a self-test mode of operation.

### DESCRIPTION OF THE INVENTION

Illustrative embodiments and exemplary applications will now be described with reference to the accompanying drawings to disclose the advantageous teachings of the present invention.

While the present invention is described herein with reference to illustrative embodiments for particular applications, it should be understood that the invention is not limited thereto. Those having ordinary skill in the art and access to the teachings provided herein will recognize additional modifications, applications, and embodiments within the scope thereof and additional fields in which the present invention would be of significant utility.

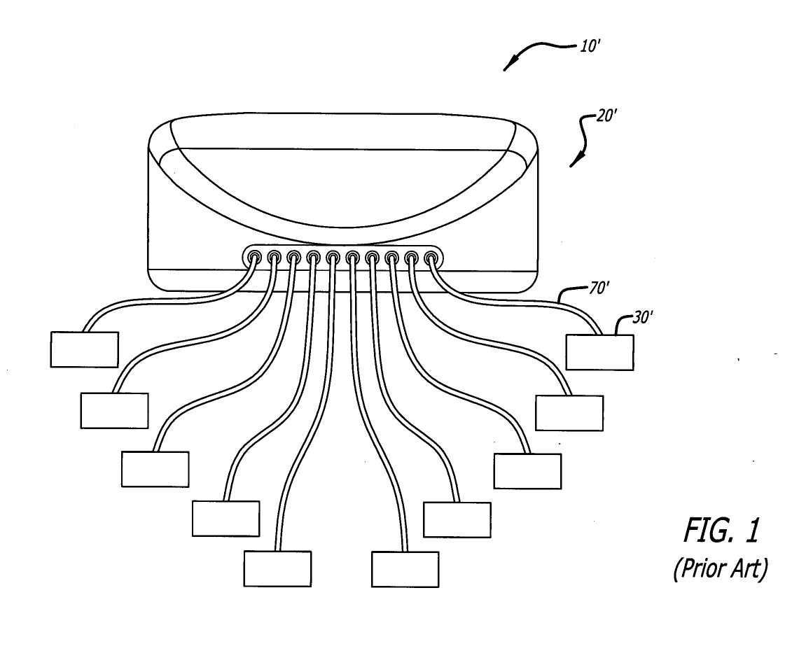

FIG. 1 is a perspective view of a typical thermostimulation system implemented in accordance with conventional teachings. The system 10′ includes a conventional thermostimulation console 20′ and a plurality of thermostimulation pads 30′. The console may be purchased from Ross Estetica of Barcelona Spain. (See http://corporativa.ross.es/rosseng/ross/indexross.htm.)

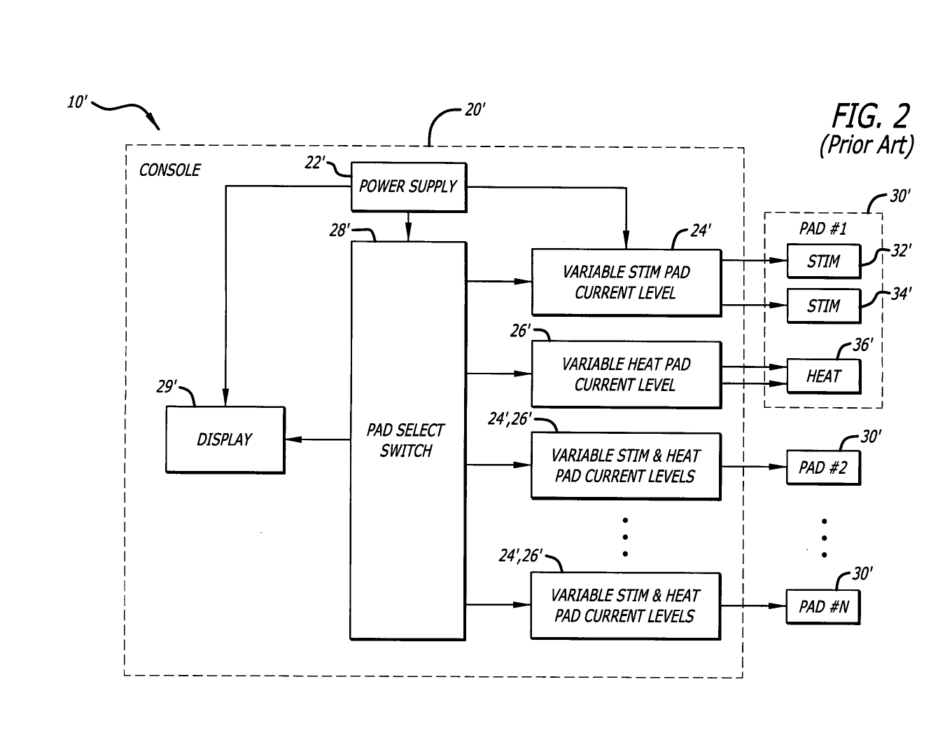

FIG. 2 is a simplified block diagram of a typical thermostimulation electrical system provided within the console of FIG. 1. The system 10′ includes a power supply 22′ disposed in the console 20′ that provides current for the pads 30′ through a set of attenuators 24′ and 26′ for each pad 30′. The first attenuator 24′ regulates current to a set of stimulation contacts 32 and 34 provided on an exposed surface of the pad 30′ and the second attenuator 26′ regulates current to a heating coil 36′ embedded within the pad. A pad select switch 28′ provides an enable signal for each attenuator under operator control and outputs the setting level status to the operator via a display 29′. Note that the system 10′ only sets the heat and stimulation current levels.

The therapeutic method enabled by the conventional system 10′ includes the steps of:
1. The system 20′ is switched on by the main switch located at the back of the device.
2. Press the on/off button located at the front, a green light is shown and a “beep” sound indicates this.
3. Press the +button next to “program” and continue pressing until the program you want comes on the screen.
4. Begin pre-heat by pressing the start button then heat button. The machine will automatically start to heat the pads. This takes approximately 3 minutes.
5. Once pre-heat has ended the system 20′ will “beep” once when ready for stimulation. Consultant pauses machine once required heat is achieved on pads using the “pause” button. Press pause again to start the treatment at full 45 minutes.
6. Tell the patient to remove flip-flops and gown. For females give the patient a towel to cover her upper torso.
7. Tell the patient to sit on the appropriate (CD) pads (if any), and to lie back slowly onto the (B) pad (if any). Indicate to patient heat is on but no current.
8. Place bolster under the popliteal spaces of the knees.
9. Strapping: Get patient to bend one knee, place the long rectangle pad around the thigh. Secure using one of the medium straps across the centre of the pad. Place two other medium straps above and below to secure. Repeat for other leg. This is an example of how to strap one pad to a body part. (Refer to LZHR-09 cTEMs Operating Protocol)
10. Place the other pads at the appropriate parts of the body and secure.
11. When the patient is safely strapped up, press the “pause” button to start the program and then the “+current” button slowly to increase the stimulation.
12. Continue to press the +button next to “current” to start deepening the sensation, but only increase to a level that feels comfortable to the patient. This increases the current intensity for all 10 pads.
13. To increase individual pads, press the letter(s) A-J (a horizontal line will appear above the letter) and then the + next to the “current” button and likewise for decreasing, use the − button.
   NB. Once the consultant has pressed the desired letter, if the +current button is not pressed, it will automatically be reset. The consultant will then have to press the letter again to activate it and then increase/decrease the intensity.
14. Once the patient threshold is reached, maintain levels, increase/decrease intensity accordingly until end of treatment.
15. During the treatment the consultant fills in the Patient Lifestyle Sheet located in Patient Consultation Form.
16. Patient percentage intensities are recorded on Patient Intensity Sheet. Patient pad layout is recorded on Patient Pad Layout Sheet located in patient consultant forms.
17. Once the treatment is over the machine will make a single “beep” noise. The machine can then be turned off from the switch at the front and then the main power switch at the back.
18. Unstrap the patient and get the patient to sit on edge of bed. Post treatment information received to how they are feeling, any adverse effects. If any an Incident Report is generated. Input of: Name, Date of Incident, What happened, What was done, Consultant Signature is recorded.
19. Post-treatment advice for patient to drink plenty of water, go to the bathroom and have shower. Walk patient to changing room.

As no temperature sensor is provided in the conventional pad 30′, no pad temperature regulation or control is possible with the system 10′.

As noted above, copending U.S. patent applications entitled THERMOSTIMULATION SYSTEM INCLUDING MULTILAYER PADS WITH INTEGRATED TEMPERATURE REGULATION, filed ______ by L. Mohn (Atty. Docket No. Luzmon-1), THERMOSTIMULATION PAD WITH INTEGRATED TEMPERTURE SENSOR, filed ______ by L. Mohn (Atty. Docket No. Luzmon-6), and INLINE CONTROL SYSTEM FOR THERAPEUTIC PAD, filed ______ by L. Mohn (Atty. Docket No. Luzmon-7), the teachings of each of which are incorporated herein by reference, disclose and claim a novel system and apparatus for thermostimulation using both thermal and electrical stimulation in inline control and pads with temperature feedback.

Hence, a need remains in the art for new therapeutic methods that leverage the capabilities of the system and apparatus taught by Mohn in the above-noted applications. As discussed more fully below, the inventive method addresses this need.

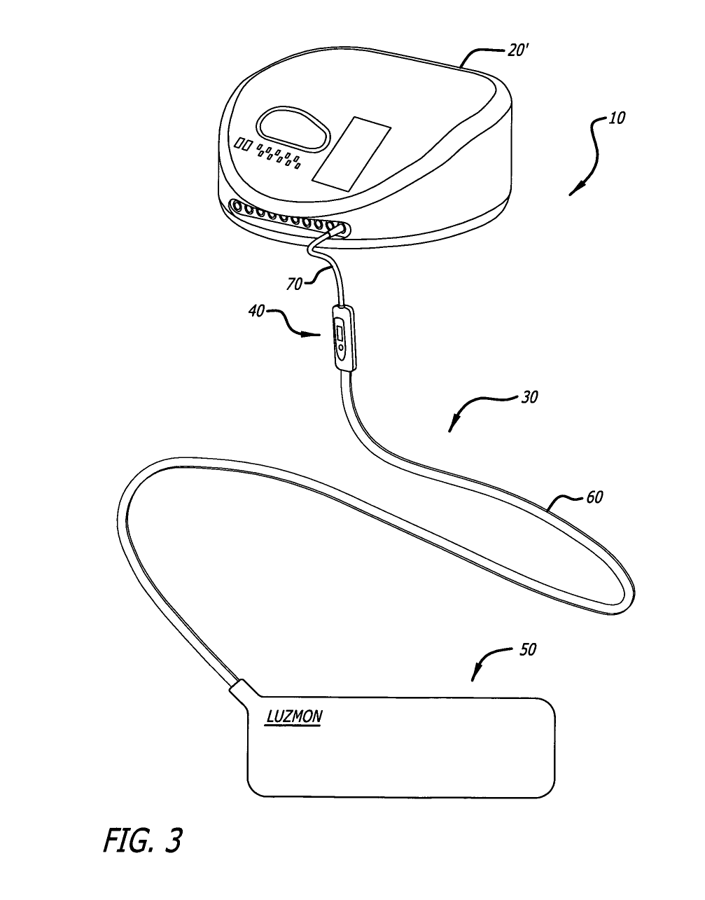

FIG. 3 is a simplified perspective view of a thermostimulation system implemented in accordance with an illustrative embodiment of the teachings of the above-noted copending application entitled THERMOSTIMULATION SYSTEM INCLUDING MULTILAYER PADS WITH INTEGRATED TEMPERATURE REGULATION incorporated herein by reference. As shown in FIG. 3, the system 10 includes a conventional thermostimulation console 20′ with a plurality of novel thermostimulation pad assemblies 30 electrically coupled thereto. Each pad assembly 30 includes a novel inline control system 40 and an associated multilayer injection molded dual function (heat and stimulation) pad 50 of unique design and construction with integrated sensors. Each control system 40 is connected to an associated pad 50 via a cable 60.

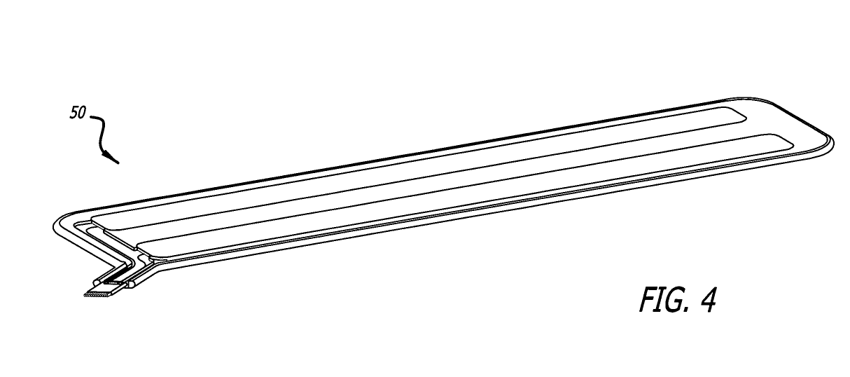

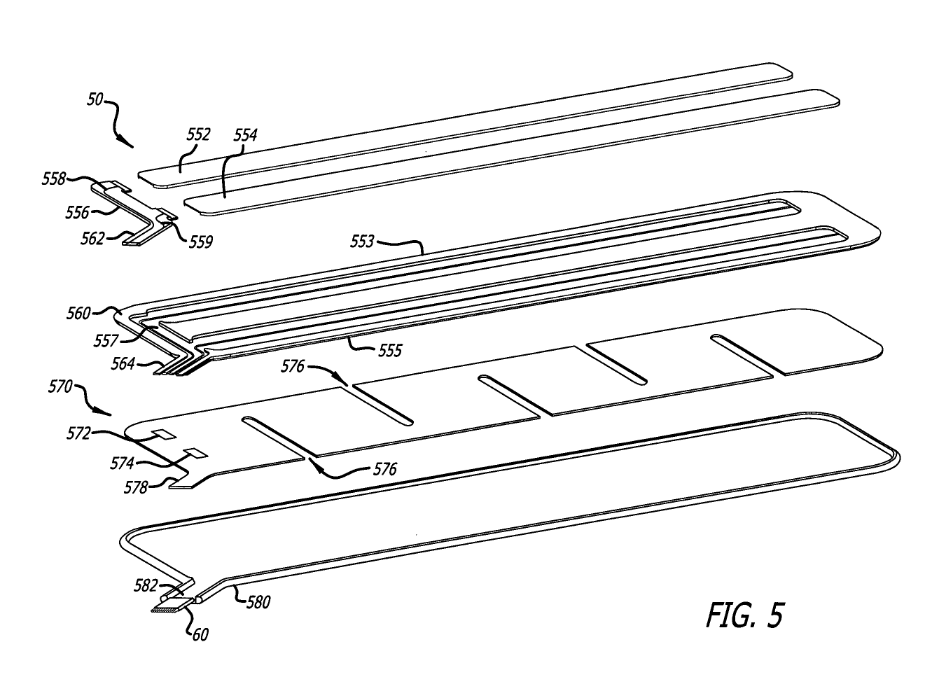

FIG. 4 shows a perspective bottom view of the pad 50 of FIG. 3. FIG. 5 is an exploded upside down view of a portion of the pad 50 of FIG. 3 in disassembled relation. As shown in FIGS. 4 and 5, the pad 50 includes first and second elongate substantially parallel conductive strips 552 and 554. In practice, one of the strips is a positive contact and the other provides a negative contact.

As shown in FIG. 5, a heating element 570 is included in each pad 50. In the best mode, the heating element 570 is implemented as a built-in wire matrix and is held in place with a layer of silicone 580. First and second temperature sensors 572 and 574 are mounted in the heating element 570, one is a live sensor measuring temperature and feeding this information back to the control box and the second is a back up should the first sensor fail.

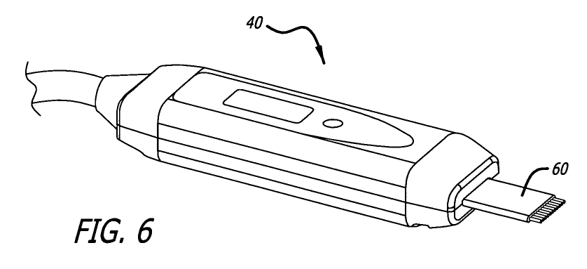

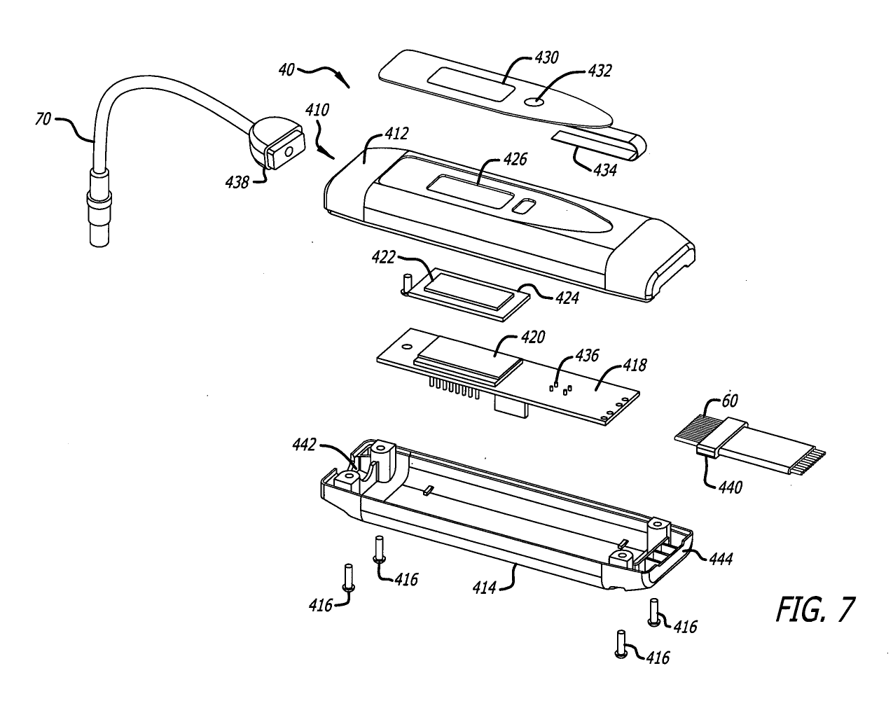

FIG. 6 is a perspective side view of the inline control system 40 of FIG. 3 fully assembled. FIG. 7 is a perspective side view of the inline control system 40 of FIG. 6 disassembled. As shown in FIG. 7, the control system 40 includes a two part injected molded ABS plastic housing 410 with an upper casing 412 and a lower casing 414. The housing 410 is adapted to retain a multilayer printed circuit board 418 on which an integrated circuit 420 is disposed. A microprocessor (not shown) is provided by the integrated circuit 420. Numerous additional electrical components are mounted onto the printed circuit board 418 along with a liquid crystal display (LCD) 422.

As shown in FIG. 7, the LCD 422 is protected by a small Perspex window 424. In the best mode, the LCD display 422 shows both the target and actual temperatures for the associated pad. The window 424 seats within an aperture 426 in the upper casing 412 of the housing 410. A plate 430 contoured to fit within a depression on the upper surface of the upper casing 412 is fitted with a manual override switch 432. In the illustrative embodiment, a switch 432 connects to the control circuitry on the printed circuit board 418 via a flexible wire 434 and pins 436. The switch enables a user to confirm when a user wants to heat a pad 50 above 38 degrees Celsius.

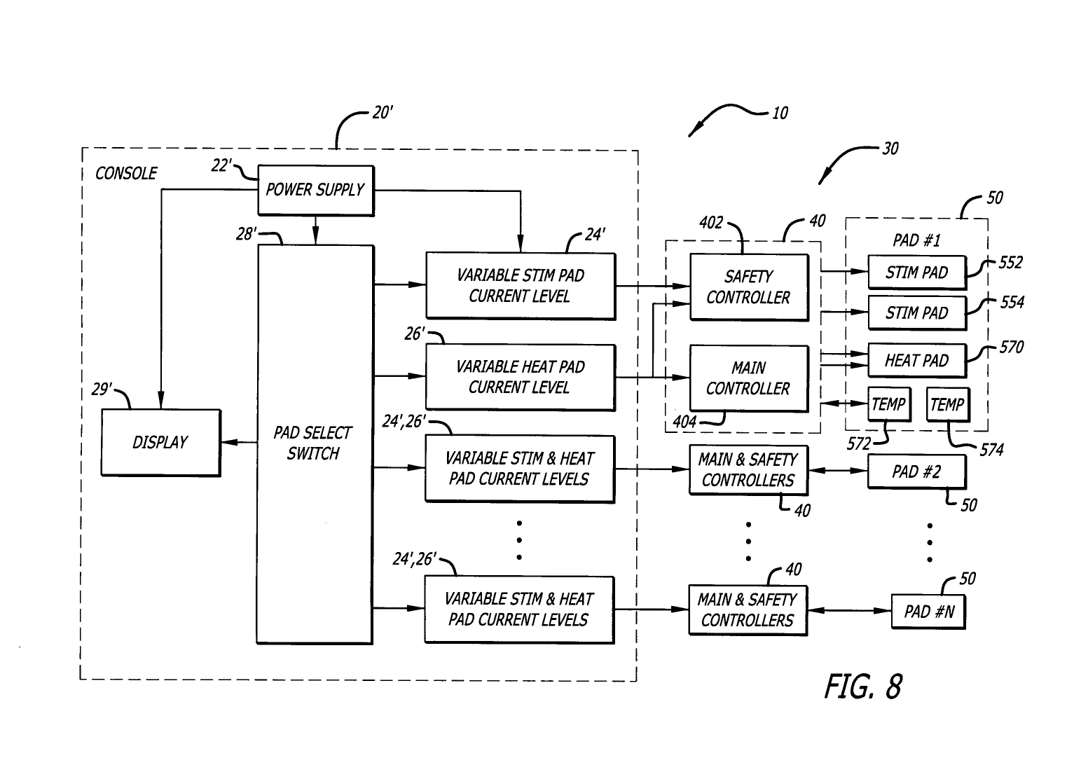

FIG. 8 is an electrical block diagram of the system 10 described in copending U.S. patent application entitled THERMOSTIMULATION SYSTEM INCLUDING MULTILAYER PADS WITH INTEGRATED TEMPERATURE REGULATION the teachings of which have been incorporated herein by reference. The control system 40 is powered by heating current from the console 20′. The control system 40 provides intelligent operation for the pad 50, monitoring the current going to both electrostimulation pads 552 and 554 and the heating element 570. These currents can be set at different levels by the control system 40 depending on the program selected or manually adjusted after a program is selected. The conventional console 20′ does not allow for the temperature to be measured or monitored but instead typically has a heating current level setting described as a “heating percentage”. Since a regulation or control functionality is not conventionally available, the current sent to the pads could allow them to heat to more than 42 degrees Celsius, a level which is outside of safe levels and the requirements set by the EN60601-2-35 standard.

As illustrated in FIG. 8, each control system 40 is implemented with first and second microcontrollers (implemented in the best mode with microprocessors) 404 and 402, that control and interrupt the current to the stimulation electrodes 552 and 554 and the heating element 570 of FIG. 6 respectively. The first controller 404 serves as a main controller and the second controller 402 serves as a safety controller. As discussed more fully below, each microcontroller runs unique software (i.e. firmware) stored on a tangible medium, such as an electrically erasable programmable read only memory (EPROM), in the integrated circuit 420 of FIG. 7.

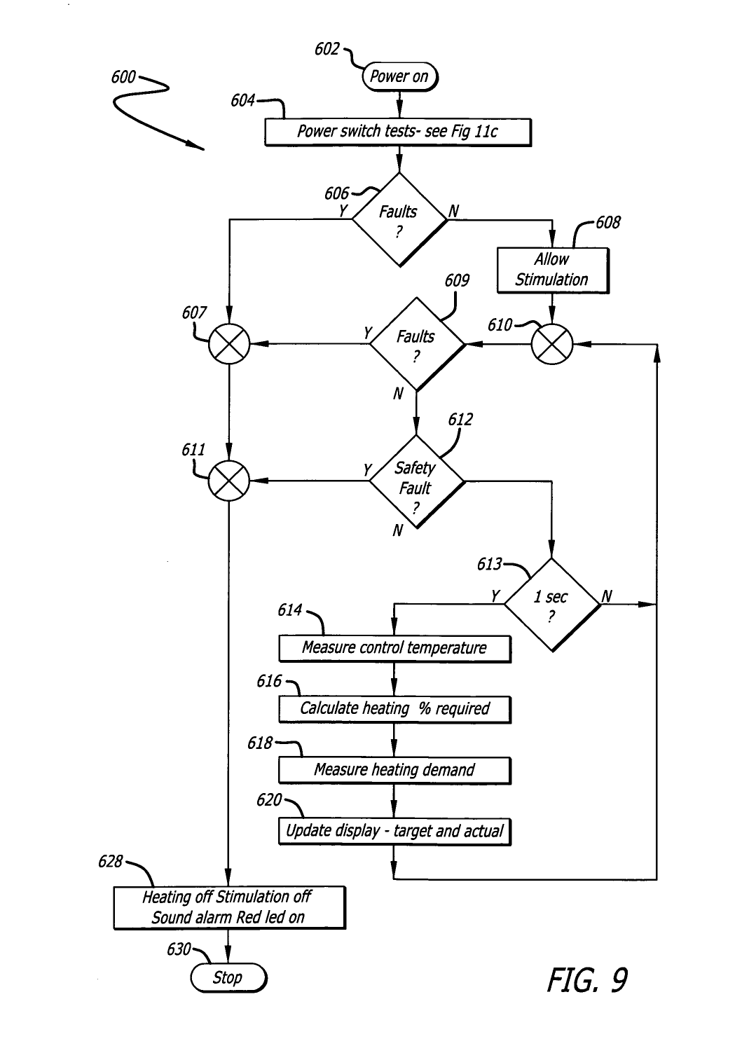

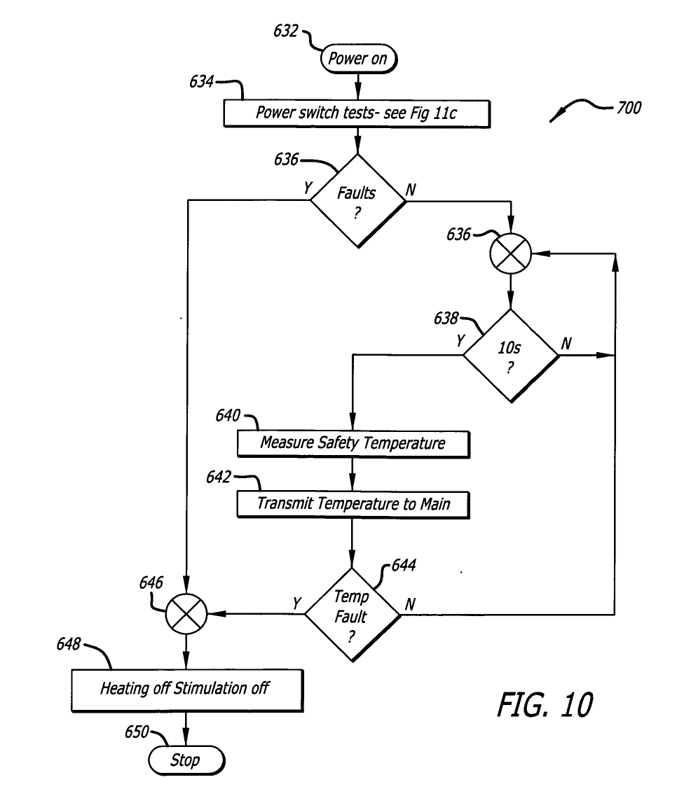

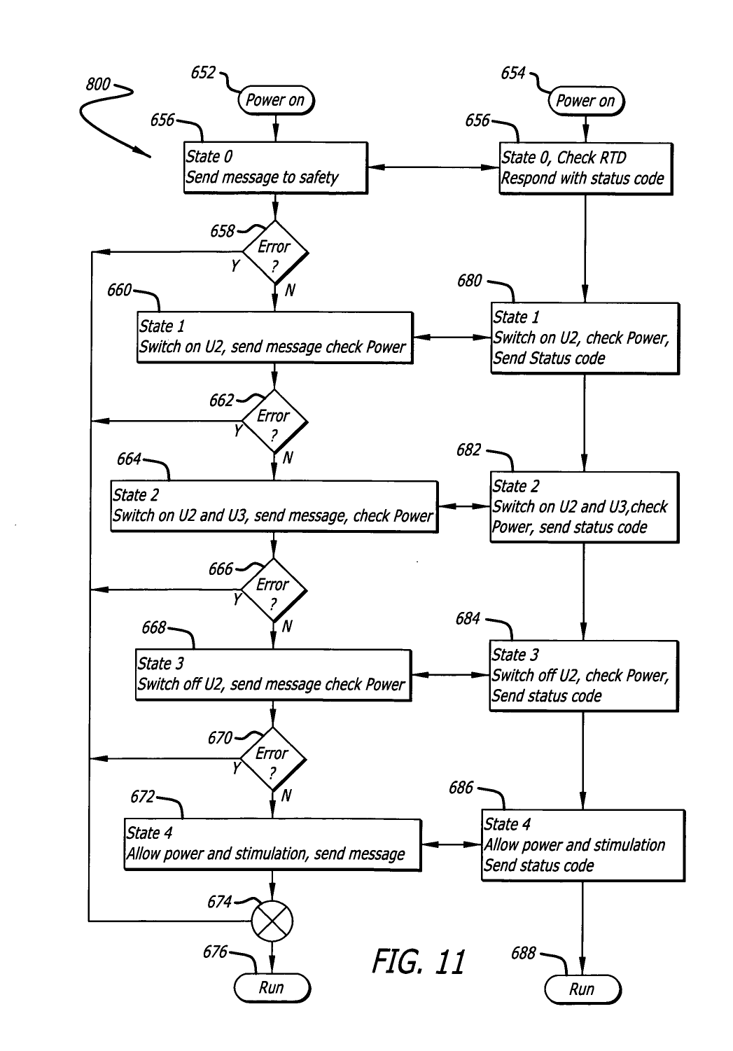

FIGS. 9-11 are flow diagrams of the firmware executed by the microprocessors in accordance with an illustrative embodiment of the present teachings. FIG. 9 is a flow diagram of the firmware executed by the main microcontroller 404 of FIG. 8. FIG. 10 is a flow diagram of the firmware executed by the safety microcontroller 402 of FIG. 8. FIG. 11 is a flow diagram of the firmware executed by the main and safety microcontrollers of FIG. 8 for a self-test mode of operation. Both microcontrollers monitor the heating power control devices to determine whether they perform the correct on-off switching action or have failed as a short circuit or an open circuit. During the power up stage, the MMC and SMC communicate using an asynchronous communications link. In the illustrative embodiment, the microprocessors communicate with each other every second to pass status information using an I2C serial interface.

The MMC 404 sends messages to the SMC to tell it which test is being performed and then the SMC 402 sends the results of the tests at each stage. Only if all the stages pass with no failures is power applied to the heating circuit 570 in the pad.

During power up (602), or at a power setting greater than five percent (5%) of maximum, the main microcontroller (MMC) 404 performs a self-test (604) to detect any possible failures and then communicates with the safety microcontroller (SMC) 402. As illustrated in FIG. 11, the self-tests are synchronized such that all hardware functionality is tested before enabling heating power to the patient.

The pad assembly, including the electronics, is calibrated. Calibration information is stored in an EPROM (not shown) within the MMC 404. In order that the SMC 402 can accurately determine whether the associated regulated pad is overheating, a calibrated maximum temperature value is passed from the MMC to the SMC during the power up procedure.

After checking for faults (606) the MMC 404 enables stimulation (608) and monitors the percentage power setting of the console 20 (see steps 614-616). This is used to set a target temperature for the pad. This target temperature is displayed on the LCD 422. Should the target temperature be greater than 38° C. the software 600 requires the operator to press the front panel switch on the console 20 to confirm the intention to set a higher temperature. Table I below lists illustrative target temperatures corresponding to various power levels.

TABLE I

| | Target |
|---|---|
| Power setting % | Temperature |
| 5-20 | 36 |
| 20-30 | 37 |
| 30-40 | 38 |
| 40-50 | 39 |
| 50-60 | 40 |
| 60-100 | 41 |

In the illustrative embodiment, a reduction in target temperature would not have to be confirmed.

During a pre-heating stage, the console unit 20′ will go to 100% for a three minute period. This is to heat up the pads prior to placement on a patient. This is interpreted as a demand for 41° C. and if this temperature is not confirmed by the operator the unit will heat up to (38° C.).

The MMC controls the temperature using a PID control loop. The actual temperature is measured using the temperature sensor 572 embedded in the pad. The SMC monitors the pad temperature using the other temperature sensor 574.

There is a two colour LED in the front facing section of the connection box. This will flash red and green and is used to provide status information.

TABLE II

| LED | Information |
|---|---|
| Green flashing | Heating up to target |
| Green continuous | Target reached |
| Red flashing | Over temperature, when actual is |
| | above target but not above 42° C. |
| Red continuous | Fault, heating and stimulation |
| | disabled. This could be temperature |
| | above 42° C. or a hardware fault. |

As shown in FIG. 10, after performing self-tests (634) the SMC 402 measures the safety temperature via the second sensor 574 and disables the associated pad 50 if the specified maximum temperature is reached or exceeded.

In a typical application, one or more silicone pads 50 are placed on the patient's body and a contact gel is used to improve electrical contact for the stimulation signals. Each pad is connected to the central console 20′ (cTEMS system) via the electrical cable 70, inline control unit 40 and connecting cable 60. The dual temperature sensors 572, 574 in each pad 50 provide temperature readings back to the inline control unit 40. The inline control unit 40 displays both the actual pad temperature and the target temperature requested by the cTEMS system (20′) on the LCD panel 422. As noted above, the cTEMS system provides electrical stimulation current through the inline control unit to the pad. Each inline control unit 40 contains two microprocessors (main controller and safety controller) and software. The main controller sends commands and waits for response from the safety controller. The main controller will shut down the power to the pad if there is no response from the safety controller. During power up the control unit will perform a start-up safety sequence. Should a fault be found, no power will be connected to the pad. The control unit software measures the heat and stimulation demand from the cTEMS system and the actual pad temperature from dual temperature sensors in the pad. The control unit has a safety feature that cuts heating to the pad if any one of the pad temperature sensors should fail.

Next, the operator selects the desired program and starts the preheating phase. The control unit will not permit the pad temperature to exceed 41° C. The control unit contains a button that must be pressed by the operator to manually confirm that the pad temperature may increase beyond a specified safety temperature. The display will flash at 41° C. and then heat up to 38° C. unless the override switch 432 (FIG. 8) is pressed at which stage it would heat to 41 C. The preheating phase lasts for 3 minutes (and can be repeated).

Once the preheating is complete, the user presses “pause” on the console 20′ and the LCD display on the inline controller 40 will go blank. The patient is laid on the bed and the pads are strapped to the patient in the desired configuration. The operator then presses pause again and the program starts. The LCD 422 will then show the actual and target temperature again and the user will have to press the switch 432 for each pad if the system program has a current percentage of 40% or above to allow the pad to heat to above 38° C. The inline controller 40 for each pad will then monitor the temperature and ensure that it does not go above the desired level. If the LED goes continually red for a period of more than a couple of minutes pad control system 40 will interrupt all the currents (both electrical stimulation and heat) to the pad and the user will put the system on pause and replace the pad.

In accordance with the present teachings, the following novel nominal therapeutic method is provided by the system 10:
1. The system 20′ is switched on by the main switch located at the back of the device.
2. Press the on/off button located at the front, a green light is shown and a “beep” sound indicates this.
3. Press the +button next to “program” and continue pressing until the program you want comes on the screen.
4. Begin pre-heat by pressing the start button then heat button. The machine will automatically start to heat the pads. This takes approximately 3 minutes.
5. Once pre-heat has ended the system 20′ will “beep” once when ready for stimulation. Consultant pauses machine once required heat is achieved on pads using the “pause” button. Press pause again to start the treatment at full 45 minutes.
6. After preheating, the temperature is automatically maintained by the inline controllers 40.
7. Tell the patient to remove flip-flops and gown. For females give the patient a towel to cover her upper torso.
8. Tell the patient to sit on the appropriate (CD) pads (if any), and to lie back slowly onto the (B) pad (if any). Indicate to patient heat is on but no current.
9. Place bolster under the popliteal spaces of the knees.
10. Strapping: Get patient to bend one knee, place the long rectangle pad around the thigh. Secure using one of the medium straps across the centre of the pad. Place two other medium straps above and below to secure. Repeat for other leg. This is an example of how to strap one pad to a body part. (Refer to LZHR-09 cTEMs Operating Protocol)
11. Place the other pads at the appropriate parts of the body and secure.
12. When the patient is safely strapped up, press the “pause” button to start the programme and then the “+current” button slowly to increase the stimulation.
13. Continue to press the +button next to “current” to start deepening the sensation, but only increase to a level that feels comfortable to the patient. This increases the current intensity for all 10 pads.
14. To increase individual pads, press the letter(s) A-J (a horizontal line will appear above the letter) and then the + next to the “current” button and likewise for decreasing, use the − button.
   NB. Once the consultant has pressed the desired letter, if the +current button is not pressed, it will automatically be reset. The consultant will then have to press the letter again to activate it and then increase/decrease the intensity.
15. Once the patient threshold is reached, the inline controllers 40 automatically maintain levels, increase/decrease intensity accordingly until end of treatment.
16. During the treatment the consultant fills in the Patient Lifestyle Sheet located in Patient Consultation Form.
17. Patient percentage intensities are recorded on Patient Intensity Sheet. Patient pad layout is recorded on Patient Pad Layout Sheet located in patient consultant forms.
18. once the treatment is over the machine will make a single “beep” noise. The machine can then be turned off from the switch at the front and then the main power switch at the back.
19. Unstrap the patient and get the consultant to sit on edge of bed. Post treatment information received to how they are feeling, any adverse effects. If any an Incident Report is generated. Input of: Name, Date of Incident, What happened, What was done, Consultant Signature is recorded.
20. Post-treatment advice for patient to drink plenty of water, go to the bathroom and have shower. Walk patient to changing room.

In addition, in accordance with the present teachings, the following novel alternative therapeutic methods are provided by the system 10:
a. Heating of a body region for the relief of minor muscular or joint pain;
b. Relaxation of muscle spasms;
c. Prevention or retardation of disuse atrophy;
d. Increasing local blood circulation;
e. Muscle re-education;
f. Post-surgical stimulation of calf muscles to prevent veneous thrombosis; and
g. Maintaining or increasing range of motion;
h. Management of chronic, intractable pain
i. Post-traumatic acute pain
j. Post-surgical acute pain

Thus, the present invention has been described herein with reference to a particular embodiment for a particular application. Those having ordinary skill in the art and access to the present teachings will recognize additional modifications, applications and embodiments within the scope thereof. For example, the pad may contain galvanic skin response (GSR) sensors in addition to the temperature sensors. The GSR sensors measure skin conductivity and provide readings to the inline control unit. The inline control unit may pass these GSR readings to the console 20′ which may use the GSR readings in the treatment of dermatological conditions, or for relaxation and desensitization training.

As another alternative, the pad may contain Electromyography (EMG) sensors to measure electrical activity given off by the patient's muscles. In this case, the EMG readings are provided to the pad's inline control unit. The inline control unit adjusts the heat and/or stimulation levels based on the values from the EMG readings. The inline control unit provides the EMG readings to the console 20′. The console 20′ adjusts the heat and/or stimulation levels to each pad based on the EMG readings. The EMG readings are displayed on the LCD display of the inline control unit or the central device console 20′. The EMG readings may be used in the treatment of patients who have symptoms of weakness and/or impaired muscle strength, gait analysis.

In another embodiment, the pad contains a pulse sensor to measure the patient's heart rate. The pulse sensor passes readings back to the inline control unit. The inline control unit displays the individual pad's pulse rate on the control unit's LCD display. The inline control unit may pass the pulse rate back to the console 20′.

Each inline control unit may be adapted with a device (e.g. Bluetooth or 802.11) to communicate with other inline control units. Further, each inline control unit may include wireless features to allow communication with other external devices.

It is therefore intended by the appended claims to cover any and all such applications, modifications and embodiments within the scope of the present invention.

Accordingly,

What is claimed is:

1. A therapeutic method including the steps of:
applying a pad to a patient having a biometric sensor adapted to feedback a signal;
coupling said feedback signal to an inline control system;
coupling said inline control system to a console; and
regulating energy applied to said pad by the inline control system based on the output of said sensor coupled to said inline control system and an output of said console.

2. The invention of claim 1 wherein said therapeutic method includes the steps of applying heat via said pad.

3. The invention of claim 2 wherein said sensor is a temperature sensor.

4. The invention of claim 3 wherein said therapeutic method includes the step of applying electrical stimulation via said pad.

5. The invention of claim 1 wherein said therapeutic method includes the step of applying electrical stimulation via said pad.

6. The invention of claim 1 further including the step of measuring skin conductivity

7. The invention of claim 6 wherein said step of measuring skin conductivity includes the step of measuring skin conductivity with a galvanic skin response sensor embedded within said pad.

8. The invention of claim 7 further including the step of inputting the galvanic skin response sensor output to the inline control system.

9. The invention of claim 8 further including the step using galvanic skin response sensor output in the treatment of dermatological conditions.

10. The invention of claim 9 further including the step using galvanic skin response sensor output in a relaxation treatment.

11. The invention of claim 10 further including the step using galvanic skin response sensor output for desensitization training.

12. The invention of claim 1 further including the step of measuring muscular electrical activity.

13. The invention of claim 12 wherein the step of measuring muscular activity includes the step of measuring muscular activity with an electromyography sensor embedded within said pad.

14. The invention of claim 13 further including the step of coupling the output of the electromyography sensor to the inline control system.

15. The invention of claim 14 wherein said inline control system adjusts the heat and/or stimulation levels based on the output from the electromyography sensor.

16. The invention of claim 15 wherein the inline control unit provides data to the console based on the output from the electromyography sensor.

17. The invention of claim 16 wherein the output from the electromyography sensor is displayed on a display of the inline control system.

18. The invention of claim 17 wherein the output from the electromyography sensor is displayed on the console.

19. The invention of claim 18 further including the step of using the wherein the output from the electromyography sensor in the treatment of patients for weakness, impaired muscle strength, or gait analysis.

20. The invention of claim 1 further including the step of measuring a patient's heart rate.

21. The invention of claim 20 wherein the step of measuring a patient's heart rate includes the use of a pulse sensor mounted within said pad.

22. The invention of claim 21 wherein an output of the pulse sensor is fed back to the inline control system.

23. The invention of claim 22 wherein data based on the output of said sensor is provided to the control system.

24. The invention of claim 23 wherein the output of the pulse sensor is displayed on a display on the inline control unit.

25. The invention of claim 24 wherein the output of the pulse sensor is displayed on the console.

26. The invention of claim 1 wherein each inline control unit is equipped to communicate with other inline control units.

27. The invention of claim 1 wherein each inline control unit is equipped to communicate with other external devices.

28. A thermostimulation method including the steps of:
applying a thermostimulation pad with connector integrated multilayer construction to a patient having a temperature sensor adapted to feedback a temperature signal;
coupling said pad to a console via an inline control system;
setting said console to generate predetermined electrical currents to the inline control system for thermal and electrical stimulation via a first connector; and
regulating the temperature of said pad via the inline control system in response to said predetermined electrical current for thermal stimulation and said feedback temperature signal.
